(19) United States
(12) Patent Application Publication
(10) Pub. No.: US 2010/0121292 A1
(43) Pub. Date: May 13, 2010

(54) POTTY TRAINING INSERT FOR DIAPER AND COMBINATION THEREOF

(76) Inventors: Abdeslem Wakrim, Houston, TX (US); Mustapha Laribi, Brooklyn, NY (US); Abderrahim Lajfar, Houston, TX (US)

Correspondence Address: Intellipex PLLC, 30200 Telegraph Road, Suite 245, Bingham Farms, MI, 48025, US

(21) Appl. No.: 12/611,529

(22) Filed: Nov. 3, 2009

Related U.S. Application Data
(60) Provisional application No. 61/113,795, filed on Nov. 12, 2008.

(51) Int. Cl. A61F 13/15

(52) U.S. Cl. 604/361

(57) ABSTRACT

A potty training insert for a diaper and a combination thereof is provided. The potty training insert comprises a sensor and a detector. The sensor is adapted to be coupled to the diaper. The sensor comprises a pair of electrically conductive cloth strips adjacent to each other and a pair of insulative layers sandwiching the pair of electrically conductive cloth strips. The detector is operatively coupled to the pair of electrically conductive cloth strips of the sensor. The detector is adapted to produce a customized alarm signal when the pair of electrically conductive cloth strips senses moisture in the diaper. The customized alarm signal enables a child to restrain excretory movements for becoming potty trained.

### CROSS REFERENCE TO RELATED APPLICATIONS

The present disclosure claims priority under 35 United States Code, Section 119 on the U.S. Provisional Patent Application No. 61/113,795 filed on 12 Nov. 2008 the disclosure of which is incorporated by reference.

### FIELD OF THE DISCLOSURE

The present disclosure generally relates to diapers, and, more particularly to a potty training insert for a diaper and a combination thereof which assist in potty training a child.

### BACKGROUND OF THE DISCLOSURE

One of the major problems generally faced by parents and caregivers while taking care of small children is taking care of a diaper. For example, when a child accidentally urinates or defecates in a diaper, the diaper becomes wet, and the wet diaper needs to be quickly changed in order to prevent the child from acquiring skin conditions or diaper leakage. Therefore, the parents and the caregivers are required to continuously monitor the dry state of the child's diaper.

The process of continuously monitoring the dry state of small children's diaper is not desirable and accordingly the parents and the caregivers expect and desire their children to be potty trained at an early age. Specifically, the parents and the caregivers expect their children to know how to restrain excretory movements. Therefore, the parents and the caregivers provide potty training to their children at an early age. During potty training, the parents and the caregivers utilize various methods and techniques to enhance mental capabilities of a child which allows the child to learn proper bathroom habits and to become potty trained. However, potty training a child is a long and arduous process for both the parents or caregivers, and the child.

There have been other devices that have attempted similar results, such as U.S. Pat. Nos. 5,817,079 to Fard and 6,603,403 B2 to Jeutter et al., however these devices are not intended to be removable. This severely limits their usability only those diapers that can be bought with the device already attached. A removable device enables any number of diapers to be utilized, varying from size to type, such as cloth or disposable diapers from various manufacturers. Furthermore, these devices do not disclose running the length of the diaper to capture wherever the excretion may occur.

Yet other devices, such as U.S. Pat. No. 5,469,146 to Gurler and No. 7,352,286 B2 to Chan, et al., disclose devices that must be positioned at or near the site of the excretion in order to be effective. These devices are therefore difficult to use in practical situations as prediction of where the excretions will occur are difficult at best. This is particularly difficult should the diaper shift while on the child.

Still other devices, such as U.S. Pat. No. 5,959,535 to Remsburg, have wires that extend the length of a diaper. The wires may read through to the child wearing the diaper creating uncomfortable pressure points. Furthermore, wires are known to fray and can therefore poke a diaper wearer causing further discomfort.

Yet, none of the aforementioned devices allow for user adjustability. Namely, none of the aforementioned devices disclose the ability to move the sensor to either the front or the rear of the diaper for comfort of the child. Nor do the aforementioned devices disclose a customizable alert to help the child recognize when the child has either urinated or defecated.

Accordingly, there exists a need of a potty training insert for a diaper, which assists in reducing a time length for potty training a child. Alternatively, there exists a need of a potty training diaper which assists in reducing a time length for potty training a child. Additionally, there exists a need of a potty training insert that uses a flexible conductive cloth strips. Further, there exists a need of a potty training insert that allows for the attachment of the sensor unit to either the front or the rear of the diaper for comfort of the child. Further yet, there exists a need of a potty training insert that allows for user customizable alerts for the child to recognize when it has urinated or defecated.

### SUMMARY OF THE DISCLOSURE

In view of the forgoing disadvantages inherent in the disclosed references, the general purpose of the present disclosure is to provide a potty training insert for a diaper and combination thereof, to include all advantages of the disclosed references, and to overcome the drawbacks inherent in the prior art.

Accordingly, an object of the present disclosure is to provide a potty training insert for a diaper which assists in reducing a time length for potty training a child.

Another object of the present disclosure is to provide a potty training diaper which assists in reducing a time length for potty training a child.

Yet another object of the present disclosure is to provide a potty training insert which easily transfers a training regime from one person to another.

Still another object of the present disclosure is to provide a potty training insert that minimizes the time and expense of potty training.

To achieve the above objects, in an aspect of the present disclosure, a potty training insert for a diaper is provided. The potty training insert comprises a sensor and a detector. The sensor is adapted to be coupled to the diaper. The sensor comprises a pair of electrically conductive cloth strips adjacent to each other and a pair of insulative layers sandwiching the pair of electrically conductive cloth strips. The detector is operatively coupled to the pair of electrically conductive cloth strips of the sensor. The detector is adapted to produce a customized alarm signal when the pair of electrically conductive cloth strips senses moisture in the diaper. The customized alarm signal enables a child to learn to restrain excretory movements for becoming potty trained.

In another aspect of the present disclosure, a potty training diaper for a child is provided. The potty training diaper comprises a diaper and a potty training insert adapted to be mounted on the diaper. The diaper comprises an exterior surface and an interior surface opposite to the exterior surface. The potty training insert comprises a sensor and a detector. The sensor is substantially coupled to the interior surface of the diaper with an end portion of the sensor coupled to the exterior surface of the diaper. The sensor comprises a pair of electrically conductive cloth strips adjacent to each other and a pair of insulative layers sandwiching the pair of electrically conductive cloth strips. The detector is operatively coupled to the pair of electrically conductive cloth strips of the sensor and supported between the end portion of the sensor and the exterior surface of the diaper. The detector is adapted to produce a customized alarm signal when the pair of electrically conductive cloth strips senses moisture in the diaper. The customized alarm signal enables the child to restrain excretory movements for becoming potty trained.

These together with the other aspects of the present disclosure, along with the various features of novelty that characterize the present disclosure, is pointed out with particularity in the claims annexed hereto and forms a part of the present disclosure. For a better understanding of the present disclosure, its operating advantages, and the specified object attained by its uses, reference should be made to the accompanying drawing and descriptive matter in which there are illustrated exemplary embodiments of the present disclosure.

### BRIEF DESCRIPTION OF THE DRAWINGS

The advantages and features of the present disclosure will become better understood with reference to the following detailed description and claims taken in conjunction with the accompanying drawings, and in which:

FIG. 1 illustrates a perspective view of a potty training diaper, in accordance with an embodiment of the present disclosure; and

FIG. 2 illustrates a perspective view of a potty training insert of the potty training diaper of FIG. 1, in accordance with an embodiment of the present disclosure.

Like reference numerals refer to like parts throughout the description of several views of the drawings.

### DETAILED DESCRIPTION OF THE DISCLOSURE

The exemplary embodiments described herein detail for illustrative purposes are subject to many variations in structure and design. It should be emphasized, however, that the present disclosure is not limited to a potty training insert for a diaper and a combination thereof, as shown and described. It is understood that various omissions and substitutions of equivalents are contemplated as circumstances may suggest or render expedient, but these are intended to cover the application or implementation without departing from the spirit or scope of the claims of the present disclosure. Also, it is to be understood that the phraseology and terminology used herein is for the purpose of description and should not be regarded as limiting.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

The present disclosure provides a potty training insert for a diaper which assists in reducing a time length for potty training a child. The potty training insert of the present disclosure enables a child to restrain his/her excretory movements and thereby allowing the child to become potty trained in the reduced time length. More specifically, the potty training insert is adapted to train children by engaging their attention with sounds and alerts when the children accidentally urinates or defecates in the diaper. The present disclosure also provides a combination of the potty training insert and the diaper, such as a potty training diaper, which also assists in reducing a time length for potty training a child. Further, the term “child” relates to others that may require potty training or reminders such as the infirm, elderly, people with disabilities, animals, or the like.

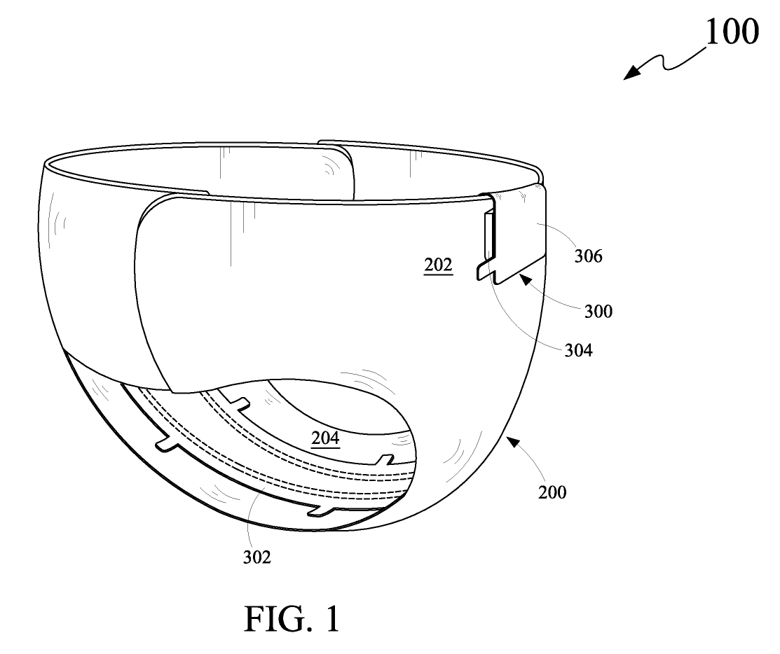

Referring to FIG. 1, a perspective view of a potty training diaper 100 is illustrated, in accordance with an embodiment of the present disclosure. The potty training diaper 100 includes a diaper 200 and a potty training insert 300 adapted to be mounted on the diaper 200. The diaper 200 includes an exterior surface 202 and an interior surface 204 opposite to the exterior surface 202. Further, the potty training insert 300 includes a sensor 302 and a detector 304 operatively coupled to the sensor 302.

The potty training insert 300 is adapted to be mounted on the diaper 200. More specifically, the sensor 302 of the potty training insert 300 is substantially coupled to the interior surface 204 of the diaper 200 with an end portion 306 of the sensor 302 is coupled to the exterior surface 202 of the diaper 200, as shown in FIG. 1. Further, the detector 304 is supported between the end portion 306 of the sensor 302 and the exterior surface 202 of the diaper 200. It should be noted that the detector 304 and the supporting end portion 306 or the sensor 302 may be positioned either in the relative front or rear of the diaper 200 without any adverse effects.

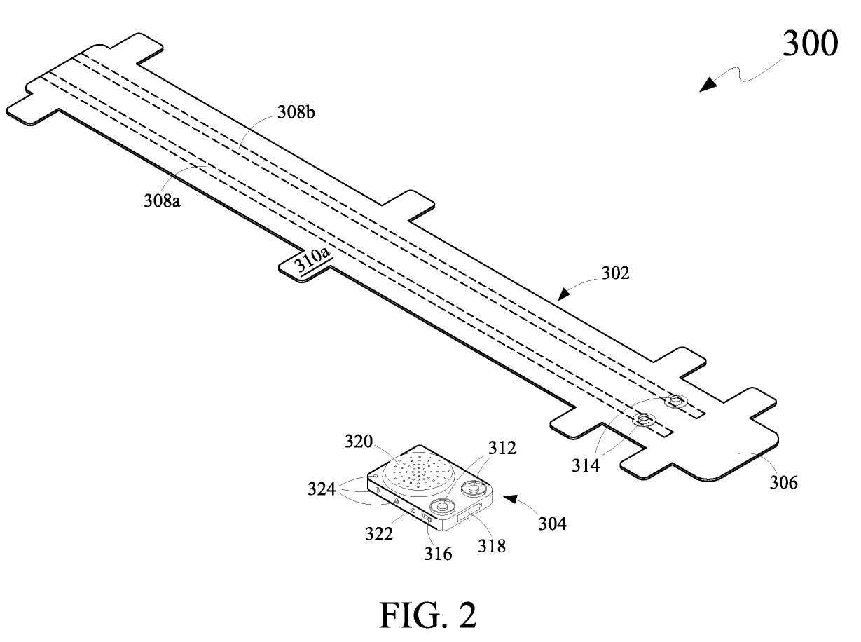

Referring now to FIG. 2, a perspective view of the potty training insert 300 of the potty training diaper 100 is illustrated, in accordance with an embodiment of the present disclosure. As shown in FIG. 2, the sensor 302 includes a pair of electrically conductive cloth strips 308a and 308b adjacent to each other, and a pair of insulative layers, such as a first insulative layer 310a and a second insulative layer (not shown), opposite to each other. It will be evident to a person skilled in the art that the second insulative layer is not shown in the FIG. 2 because the second insulative layer is positioned under the first insulative layer 310a. Further, the pair of insulative layers, such as the first insulative layer 310a and the second insulative layer, sandwiches the pair of electrically conductive cloth strips 308a and 308b therebetween. Accordingly, the pair of electrically conductive cloth strips 308a and 308b is shown with dotted lines in the FIG. 2. In the present embodiment, the use of silver impregnated cloth based conductors, such as the pair of electrically conductive cloth strips 308a and 308b, provides an advantage of an anti-bacterial component. However, it should be noted that the pair of electrically conductive cloth strips 308a and 308b, may be impregnated with other conductive materials, such as copper or the like.

Moreover, in the present embodiment, the pair of insulative layers, such as the first insulative layer 310a and the second insulative layer, is composed of a paper material. However, it may be evident to a person skilled in the art that the pair of insulative layers may be composed of other insulative materials, such as a plastic material and a rubber material. The pair of insulative layers, particularly, the first insulative layer 310a provides a barrier in avoiding a direct contact between the pair of electrically conductive cloth strips 308a and 308b with child's skin when the potty training diaper 100 is worn by a child.

As explained herein, the sensor 302 is coupled to the diaper 200. More specifically, the sensor 302 includes an adhesive layer (not show), disposed on one of the pair of insulative layers, which enables in coupling the sensor 302 to the diaper 200. In the present embodiment, the adhesive layer is disposed on the second insulative layer of the sensor 302. Accordingly, the adhesive layer enables the sensor 302 to substantially couple to the interior surface 204 of the diaper 200 and the end portion 306 of the sensor 302 to the exterior surface 202 of the diaper 200, as shown in FIG. 1. Further, the adhesive layer enables the sensor 302 to detachably couple to the diaper 200.

The sensor 302 is further operatively coupled to detector 304, as explained herein. More specifically, the detector 304 is operatively coupled to the pair of electrically conductive cloth strips 308a and 308b of the sensor 302. Further, in the present embodiment, the operative coupling of the detector 304 and the pair of electrically conductive cloth strips 308a and 308b is enabled by a snap fit arrangement having a male portion 312 and a female portion 314. The male portion 312 is configured on the detector 304 and the female portion 314 is configured on the pair of electrically conductive cloth strips 308a and 308b, as shown in FIG. 2. Accordingly, the male portion 312 is capable of being received by the female portion 314 for enabling the operative coupling of the sensor 302 and the detector 304. It should be apparent to one skilled in the art that the arrangement of male portion 312 and female portion 314 of the snaps may be of any configuration. Furthermore, it should be apparent that the snap fit connection is one style that may be utilized and other attachment mechanisms may be utilized.

In use, the detector 304 is adapted to produce a customized alarm signal when the pair of electrically conductive cloth strips 308a and 308b senses moisture in the diaper 200.

In the present embodiment, the detector 304 is an electronic device having plurality of electrical components, such as a switch 316, a battery 318, a speaker 320, and a visual indicator 322. Further, it will be evident to a person skilled in the art that the detector 304 may include electronic circuitry (not shown) adapted to electrically couple such electrical components for the controlled functions thereof. For example, the electronic circuitry may enable the switch 316 to turn ON/OFF the detector 304. Furthermore, the electronic circuitry may enable the battery 318 to power the detector 304. Moreover, the electronic circuitry may enable the speaker 320 to play the customized alarm signal and the visual indicator 322 to illuminate.

The electronic circuitry may also allow the sensor 302 to communicate with the detector 304. More specifically, the electronic circuitry of the detector 304 may communicably coupled to the pair of electrically conductive cloth strips 308a and 308b of the sensor 302 with the detector 304. Accordingly, when the child urinates or defecates in the diaper 200 the pair of electrically conductive cloth strips 308a and 308b senses the moisture in the diaper 200 and communicates to the detector 304 for allowing the speaker 320 to play the customized alarm signal and for illuminating the visual indicator 322. Further, the customized alarm signal may be played in a loop and the visual indicator 322 may illuminate until the detector 304 is turned OFF with the help of the switch 316 by a parent or a caregiver.

In the present embodiment, the customized alarm signal may be a personalized message of the parent or the caregiver. More specifically, the customized alarm signal may be a voice message of the parent or the caregiver recorded in their voices such that their child will understand and recognize the customized alarm signal. Therefore, it may be evident to a person skilled in the art that the detector 304 may further include a recording device 324 which enables the parent or the caregiver to record and replay the voice messages in their voices. However, it may be evident to a person skilled in the art that the customized alarm signal may be a melodious tune and a song that may be installed by way of a separate input, apart from the recorded voice messages of the parent or the caregiver. Additionally, the visual indicator 322 might also be customized to create patterns various light patterns or steady state illumination.

The customized alarm signal enables the child to learn to restrain his/her excretory movements and thereby allowing the child to become potty trained. More specifically, the customized alarm signal enables notification to both the parent or caregiver and the child about the excretory movements. Further, the customized alarm signal will be familiar to the child for enabling the child to recognized when he/she has gone to a bathroom and thereby helping the child to learn to restrain his/her excretory movements. Accordingly, the customized alarm signal enables in reducing a time length to potty train the child and to switch the child from using diaper to normal underwear.

Based on the foregoing description of the present disclosure, it would be apparent to those skilled in the art that a potty training insert for a diaper, such as the potty training insert 300, and a combination thereof, such as the potty training diaper 100, are capable of assisting in reducing a time length to potty train a child. The potty training insert of the present disclosure may be a partially reusable product. More specifically, a sensor, such as the sensor 302 of the potty training insert may be disposed, however a detector, such as the detector 304, may be reused in conjunction with a separate sensor. Accordingly, the potty training insert of the present disclosure provides a cost effective means for potty training the child. Further, the potty training insert of the present disclosure may be made of various sizes for being utilized with various sizes of diapers.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, to thereby enable others skilled in the art to best utilize the present disclosure and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the claims of the present disclosure.

What is claimed is:

1. A potty training insert for a diaper, the potty training insert comprising:
a sensor adapted to be coupled to the diaper, the sensor comprising
a pair of electrically conductive cloth strips adjacent to each other, and
a pair of insulative layers sandwiching the pair of electrically conductive cloth strips; and
a detector operatively coupled to the pair of electrically conductive cloth strips of the sensor, the detector adapted to produce a customized alarm signal when the pair of electrically conductive cloth strips senses moisture in the diaper, the customized alarm signal enables a child to learn to restrain excretory movements for becoming potty trained.

2. The potty training insert of claim 1, wherein the operative coupling of the detector and the pair of electrically conductive cloth strips of the sensor is enabled by a snap fit arrangement having a male portion and a female portion, the male portion is configured on the detector and the female portion is configured on the pair of electrically conductive cloth strips.

3. The potty training insert of claim 1, wherein the sensor comprises an adhesive layer disposed on one of the pair of insulative layers, the adhesive layer enables in coupling the sensor to the diaper.

4. The potty training insert of claim 1, wherein the pair of insulative layers composed of a paper material.

5. The potty training insert of claim 1, wherein the sensor is able to mounted on either a front or rear portion of the diaper.

6. A potty training diaper comprising:
a diaper having an exterior surface and an interior surface opposite to the exterior surface; and
a potty training insert adapted to be mounted on the diaper, the potty training insert comprising
a sensor substantially coupled to the interior surface of the diaper with an end portion of the sensor coupled to the exterior surface of the diaper, the sensor comprising a pair of electrically conductive cloth strips adjacent to each other and a pair of insulative layers sandwiching the pair of electrically conductive cloth strips, and
a detector operatively coupled to the pair of electrically conductive cloth strips of the sensor and supported between the end portion of the sensor and the exterior surface of the diaper, the detector adapted to produce a customized alarm signal upon the pair of electrically conductive cloth strips senses moisture in the diaper, the customized alarm signal enables a child to restrain excretory movements for becoming potty trained.

7. The potty training diaper of claim 6, wherein the operative coupling of the detector and the pair of electrically conductive cloth strips of the sensor is enabled by a snap fit arrangement having a male portion and a female portion, the male portion is configured on the detector and the female portion is configured on the pair of electrically conductive cloth strips.

8. The potty training diaper of claim 6, wherein the sensor comprises an adhesive layer disposed on one of the pair of insulative layers, the adhesive layer enables in substantially coupling the sensor to the interior surface of the diaper and the end portion of the sensor to the exterior surface of the diaper.

9. The potty training diaper of claim 6, wherein the pair of insulative layers is composed of a paper material.

10. The potty training diaper of claim 6, wherein the sensor is able to mounted on either a front or rear portion of the diaper.
